(19) United States
(12) Patent Application Publication
(10) Pub. No.: US 2023/0284670 A1
(43) Pub. Date: Sep. 14, 2023

(54) SYSTEMS AND METHODS FOR ADDITIVE MANUFACTURING OF MATERIALS WITH CONTROLLABLE MICROSCALE TEXTURES

(71) Applicant: THE REGENTS OF THE UNIVERSITY OF CALIFORNIA, Oakland, CA (US)

(72) Inventors: Boris Rubinsky, El Cerrito, CA (US); Dan Rubinsky, San Francisco, CA (US); Gideon Ukpai, Kensington, CA (US)

(73) Assignee: THE REGENTS OF THE UNIVERSITY OF CALIFORNIA, Oakland, CA (US)

(21) Appl. No.: 18/167,292

(22) Filed: Feb. 10, 2023

Related U.S. Application Data
(63) Continuation of application No. PCT/US2021/049971, filed on Sep. 10, 2021.
(60) Provisional application No. 63/077,509, filed on Sep. 11, 2020.

(51) Int. Cl. A23P 20/25

(52) U.S. Cl. CPC A23P 20/25; B33Y 10/00

(57) ABSTRACT

Systems and methods for additive manufacturing of biological matter with desired non-homogeneous and non-isotropic textures from deposited 2-D or 3-D printed elements. Desired textures, such as anisotropic structure at the microscale level, are achieved through a combination of controlled chemical, thermal and freezing steps producing crosslinked anisotropic structures by directional solidification. The apparatus has a movable printing platform associated with a heating module and a cooling module separated by a gap that creates a thermal gradient permitting directional solidification of a printed object as the platform moves over the modules.

### CROSS-REFERENCE TO RELATED APPLICATIONS

This application claims priority to, and is a 35 U.S.C. § 111(a) continuation of, PCT international application number PCT/US2021/049971 filed on Sep. 10, 2021, incorporated herein by reference in its entirety, which claims priority to, and the benefit of, U.S. provisional patent application Ser. No. 63/077,509 filed on Sep. 11, 2020, incorporated herein by reference in its entirety. Priority is claimed to each of the foregoing applications.

The above-referenced PCT international application was published as PCT International Publication No. WO 2022/056333 A1 on Mar. 17, 2022, which publication is incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This technology pertains generally to additive manufacturing fabrication techniques and methods and more particularly to systems and methods for the production of objects with controllable anisotropic, microscale textures through modulated chemical, thermal and freezing processes. The technology is particularly suited for the formation of microfibers and other textures in formulated food products.

### 2. Background

Solid freeform fabrication, also known as additive manufacturing, is a set of manufacturing processes and techniques that allows the fabrication of complex, three-dimensional structures directly from computer models and computer-aided design (CAD) data without the use of tooling such as molds or dies. Additive manufacturing fabrication techniques can produce functional, complex objects by gradually depositing material, layer by layer, on a platform substrate in an x-y plane. The platform is typically connected to a z-axis step motor that moves the platform perpendicularly to the x-y plane as the three-dimensional object is created incrementally.

Additive manufacturing can be carried out by using any of a number of processes including extrusion-based techniques utilizing a computer-controlled printing head with an extrusion tip that deposits a flow of material in an x-y plane on a substrate. The extruded material fuses to previously deposited material and solidifies upon a decrease in temperature. The position of the print head is then advanced in increments along the z-axis relative to the x-y plane of the substrate. The object is formed by repeatedly applying stable new layers on to the already formed layers.

Some of the more conventional additive manufacturing methods employ a voxel (i.e. volume element) by voxel assembly of an object under computer-controlled deposition of each voxel. The three-dimensional shape is formed when each voxel becomes bound to the other, usually through solidification or cross linking between voxels.

Food products can also be fabricated with 3-D printing using the same principle of computer-controlled voxel by voxel assembly. Multiple reservoir extrusion heads may allow combinations of stock food materials and some control over the placement of macro-nutrients, micro-nutrients, colors or flavors on the surface or within the interior of the food object. While this technique can generate biological materials and food products with heterogeneous compositions, because the assembly is voxel by voxel, the technology cannot be used to produce objects with a desired texture, in particular with microscopic (e.g. micron scale) structures. However, many foods, for example meats, are characterized by their fibrous structure and consumers seek such textures.

Accordingly, there is a need in the food industry for efficient and specialized manufacturing of biological materials for food consumption with a desired textures at the microscale level. There is also a need for the development of large scale, mass production systems and 3D printers that are capable of printing at several micron scale resolutions. Current extrusion technologies are not suitable for mass scale production of food because of the lengthy time involved in making one product.

### BRIEF SUMMARY

The present technology is directed to a 3D material printing system and solid freeform fabrication methods that allow control over microstructure and texture and are particularly suited for the preparation of a variety of edible products. The technology uses a process of directional solidification that is illustrated with directionally frozen food products manufactured by 3D printing. The use of the directional solidification element permits control over the microstructure and texture in 3D printed foods manufactured by directional 3D printing through controlled freezing.

For example, the technology and methods can generate thin slices of food products that have controlled composition as well as controlled macroscopic and microscopic textures. The directional solidification of the thin slices of food involves freezing in controlled directions such as along the long part of a thin food slice or in the direction of the thin part of a food slice. Both modalities are valuable for the production of thin food slices.

Conventional additive manufacturing employs isotropic individual volume elements (IIEV) deposited element by element to develop a 3-D structure with the IIEV elements deposited from a printer head under computer control. The preferential method is for each IIEV element to become incorporated in the 3-D structure as soon as they are deposited.

A major benefit of 3D printing is the control over the macrostructure of the object that may be achieved through IIVE by IIVE element deposition and incorporation of the additive elements (IIVE) at precise locations. In this additive manufacturing scheme, it is also possible to control the microstructure of the 3D object through freezing. Briefly, ice crystal size and orientation are major factors that may affect the microstructure of the 3D object. The ice crystal size and orientation may generally depend on the thermal history of the product during freezing. By controlling the thermal history of formation, it is possible to control the microstructure.

Accordingly, the present technology provides systems and methods that facilitate the manufacturing of macroscopic anisotropic elements with controlled anisotropic orientation by additive manufacturing; and the generation of anisotropic microscale structures by freezing in a controlled direction.

For example, a method of additive manufacturing of biological matter is provided that produces a desired texture such as with an anisotropic structure at the microscale level based on depositing the 2-D and 3-D printed elements in a preferential direction to form an anisotropic microscopic structure prior to cross-linking of the materials in the structure with either chemical methods, temperature dependent methods or electromagnetic radiation methods.

The porosity of the 3D object is another design parameter that may be controlled. Generally, the porosity of printed structure may be a key parameter in the product design. One method for producing pores is by freezing and then freeze-drying a thickening agent such as a gel, e.g., hydrogel solution. The eventual size, direction, and shape of the pores will generally depend on the thermal parameters present during freezing.

Because ice has a tight crystallographic structure, when an aqueous solution freezes the solutes are typically rejected by the ice front while the ice crystals are made of pure water. Constitutional supercooling may cause the ice front to become dendritic (fingerlike) in the direction of propagation, potentially entrapping solutes between the ice crystals. After freeze-drying, the ice crystal sites form the pores and the solutes between the ice crystals may form the walls of the pore. The dimensions of the dendrites may be related, e.g. directly related, to the rate of freezing and to the amount of solutes in the solution, where higher cooling rates tend to produce smaller ice crystals.

The mode of freezing and the directionality of the freezing process may also affect the ultimate size and form of the pores created by the removal of the ice through freeze-drying. Directional solidification may be employed as a method to produce a product structure where the dimensions and the direction of the pores are controlled by controlling the direction of ice crystal propagation and the thermal history during freezing and thereby produce an object with a controlled anisotropic microscale structure from 3D printing with isotropic elements.

While cooling rates during freezing can affect ice crystal sizes during freezing, directional solidification is needed to control the direction of the ice crystals and therefore make a product, and in particular a food product with controlled anisotropic microstructure and thereby controlled texture.

In one method, directional solidification of the printed object placed on a moving stage takes place when the stage translates from the warm part of the stage to the cold part to solidify the biological matter with controlled conditions. A second method involves the deposition of the thin slice of food material on a constant temperature stage at a subfreezing temperature.

Directional solidification will generate anisotropic microstructures and food texture in the plane normal to the thin dimension of the food slice and the second method will generate anisotropic microstructures in the direction of the thin dimension of the food slice. The advantage of combining 3D printing with directional freezing is that the 3D printing can make a heterogenous material with controlled heterogeneity while the directional freezing can impart controlled anisotropic microstructure.

The preferred system for manufacturing products is based on using 3D printing technology to deposit 3-D printing elements with a selected material composition on a 3D printing surface that moves preferentially in one direction in combination with a directional solidification stage for freezing the 3D printed biological matter in such a way that ice crystals with controlled dimensions and orientations are produced. The directional freezing of the thin food slice can be either in a direction along the long part of the thin slice using directional solidification and a temperature gradient stage or in the direction of the slice thin thickness using a constant temperature stage.

Suitable materials for printing are generally compositions of aqueous solutions of biological and/or organic matter and at least one thickening agent. The deposited material may be in the form of a paste, dispersion, suspension, slurry or solution of organic matter that is printed in layered patterns determined by the printer programming. The biological matter may comprise at least one of a protein, a fat, and a carbohydrate. The biological matter may also comprise cells grown in an in vitro cell culture or particles of plant or animal tissues. The composition of deposited materials may also include at least one thickening agent, preferably selected from the group of agar, collagen, or an alginate. While these thickening agents are preferred, other equivalent agents may be used.

Although a single printer dispensing head delivering a single type of material may be described, it will be understood that the printing head can dispense more than one type of material in the formation of the 3D object. The printing methods incorporate all known 3-D printing techniques for biological matter.

The deposition of the composition is in the form of volume elements, which preferably maintain their structure temporarily before the crosslinking due to high viscosity mixtures. However, the composition may be designed to have a viscosity that is high enough to maintain its structure upon printing without the use of a crosslinking step.

The cross-linking step can be done after the deposition of the 2D or 3-D structure to capture that structure. The purpose of the initial increased viscosity is to maintain the structure under light mechanical load until the crosslinking of the entire object can take place. In another modality the crosslinking can be also done after the freezing and during the melting. In addition to generating microstructure, freezing can be also used to provide mechanical rigidity to the 3-D structure until crosslinking. The freezing will aid in maintaining the structure before the crosslinking occurs in this embodiment. Anisotropic microstructure is obtained by directional freezing in a controlled direction and with controlled thermal parameters of temperature gradient and freezing velocity.

The technology is particularly designed for manufacturing thin food products for the purpose of generating microscale anisotropy in a controlled direction and with controlled orientation to produce a desired texture in the food product. The dimensions of thin slices are preferably defined as slices in which the large dimensions are larger by a factor of between 5 and 10,000 than the small dimensions.

According to another aspect of the technology, a method is provided with the steps of preparing an aqueous solution comprising a food base, combining the aqueous solution with an edible thickening agent to produce a deposition mixture, forming the deposition mixture into a plurality of volume elements on a first surface, transferring the plurality of volume elements from the one surface to a second surface, assembling the plurality of anisotropic volume elements on the second surface in a three-dimensional array, and crosslinking the plurality of individual anisotropic elements in the three-dimensional array to produce the final food product.

The method may also comprise structurally reinforcing the plurality of individual anisotropic elements before transferring the plurality of individual anisotropic elements to the second surface. Structurally reinforcing the plurality of individual anisotropic elements may comprise freezing the plurality of individual anisotropic elements.

In some embodiments, the viscosity and texture of the food product can be selected to be suitable for the specific needs of a subject. For instance, the method may comprise selecting the viscosity and texture of the food product to be suitable for a subject with esophageal dysphagia.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a functional block diagram of a method for fabricating a product with controlled heterogeneity and controlled anisotropic microstructure according to one embodiment of the technology.

FIG. 2 is a functional block diagram of an alternative method for fabricating a product with controlled heterogeneity and controlled anisotropic microstructure according to another embodiment of the technology.

FIG. 3 is a functional block diagram of a second alternative method for fabricating a product with controlled heterogeneity and controlled anisotropic microstructure according to another embodiment of the technology.

FIG. 4 is a schematic side view of an apparatus for depositing and freezing using directional solidification. The apparatus has a thermally conductive surface placed on top of two controlled temperature bases, one maintained above and the other below the freezing temperature, separated by an insulating gap.

FIG. 5A is a schematic side view of an alternative apparatus that uses several printing, crosslinking and directional solidification stages in parallel according to one embodiment of the technology.

FIG. 5B is a top plan view of the apparatus embodiment of FIG. 5A.

FIG. 6A is a micrograph depicting directional ice structures after directional freezing showing parallel repeating ice dendrites.

FIG. 6B is a schematic side view of a directional ice structure showing dendritic ice crystals, intra-dendritic liquid and eutectic solids.

FIG. 6C is a scanning electron micrograph of directional solidification freezing of an agar gel solution as seen after freeze drying demonstrating the directionality of voids formed by ice sublimation.

FIG. 7 is a graph depicting elastic modulus of alginate strips when crosslinking before freezing compared with crosslinking after freezing.

FIG. 8 is a graph comparing maximum stress at failure of strips crosslinked before freezing with crosslinking after freezing.

### DETAILED DESCRIPTION

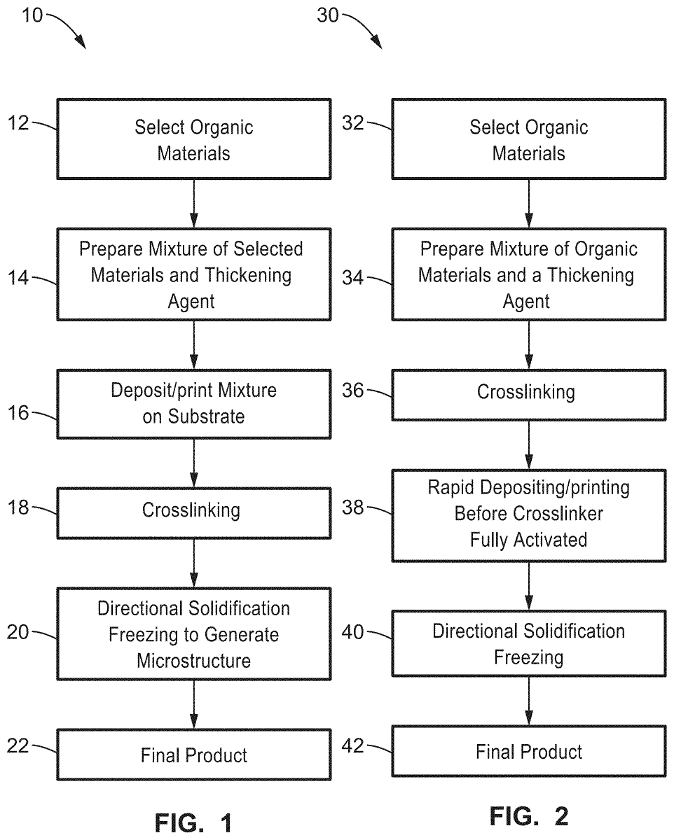

Referring more specifically to the drawings, for illustrative purposes, systems and methods for additive manufacturing of objects and materials with controllable anisotropic microscale textures are generally shown. Several embodiments of the technology are described generally in FIG. 1 to FIG. 8 to illustrate the characteristics and functionality of the devices, systems and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

A food application in the form of production of thin slices is used to generally illustrate the fabrication methods and resulting food product characteristics. In this illustration, thin slices are defined as an object that has dimension ratios of between 1 to 5 and 1 to 10,000 between one dimension of the object and the other two dimensions of the object. Although the production of a food product is used to illustrate the methods, it will be understood that the methods can be used to fabricate non-food products as well.

Turning now to FIG. 1, an embodiment of the method 10 for the additive manufacture of objects with desired anisotropic macroscopic and microscopic textures is shown schematically. At block 12, one or more types of biological matter or other organic materials to be printed are selected. Biological matter for producing food objects may include, for example, mixtures and processed mixtures of particles or cells from animal or plant sources and combinations thereof. The selected biological matter may also include various colors and flavors as well as proteins, fats and carbohydrates.

Binders or thickening agents for the deposition mixture may also be selected at block 12. One preferred thickening agent for selection at block 12 is one or more hydrogel polymers. Hydrogels can be grouped into naturally-derived polymers like collagen, alginate, agarose, chitosan, hyaluronic acid, cellulose, fibrin etc. and synthetic polymers like polyethylene glycol (PEG), polyacrylamide (PAAM), polylactic-co-glycolic acid (PLGA), polycaprolactone (PCL) etc. Ultimately the choice of thickening agents to use may depend on the application and desired product characteristics like printability, gelation kinetics, material strength, biocompatibility, control over biodegradation, and biofunctionalization.

The biological matter and one or more thickening agents are mixed with a liquid to produce a dispensing material at block 14. The deposited material may be a paste, dispersion, suspension, slurry or solution of organic matter that is printed in layered patterns determined by the printer programming.

In addition, the material to be dispensed can be selected for the type of printing mechanism to be used as well as ideal fluid properties like viscosity and surface tension that can vary from process to process. For some materials these characteristics can be obtained by varying concentration, temperature, or other processing conditions. For example, increased mechanical strength is usually associated with higher component concentrations.

In one embodiment, for example, the thickening agent may comprise agar and the method may comprise combining the aqueous solution with the agar at a temperature of greater than about 70° C. The method may further comprise assembling the anisotropic three-dimensional array at a temperature of between about 0° C. and about 40° C.

In another embodiment, the thickening agent may comprise collagen and the method may comprise combining the aqueous solution with the collagen at a temperature of between about 0° C. and about 10° C. Solidifying the plurality of individual anisotropic microscale elements in the three-dimensional array may comprise increasing the temperature of the assembled plurality of individual anisotropic elements to a temperature of between about 20° C. and about 40° C.

The mixed dispensing material prepared at block 14 is then printed or deposited on a substrate at block 16 of FIG. 1. There are a variety of additive printing technologies that may be used and adapted to produce a particular product.

After deposition of the organic or biological material at block 16, the deposited structure is cross-linked at block 18 of FIG. 1. Crosslinking is generally a process of linking the polymer chains together in ionic or covalent bonds to form a network structure that is generally stiffer. The crosslinking of hydrogels, for example, can be initiated by several means with chemical additives, photoinitiation, and temperature change being the most common.

Crosslinking the printed product at block 18 can also occur through the addition of the cross-linker as a spray on the surface of the printed product. The technique can be used with heat crosslinking for collagen or with UV crosslinking with collagen or with cross-linkers such as calcium carbonate and D-Gluconic acid δ-lactone for alginate.

For example, the method may comprise preparing an aqueous solution comprising organic matter, combining the aqueous solution with a thickening agent to produce a deposition mixture, forming the deposition mixture into a plurality of microscale anisotropic structural elements and binding the plurality of anisotropic elements into a three-dimensional array at block 18, thereby producing a product with non-homogeneous and non-isotropic textures.

In another embodiment, the selected thickening agent comprises an alginate and the deposition mixture is combined with calcium carbonate and D-Gluconic acid δ-lactone followed by rapid deposition of the combination in an anisotropic structure before the crosslinking has affected the entire mixture. The activated thickening agent solidifies the plurality of individual anisotropic elements into a three-dimensional array.

In some embodiments, the sodium alginate thickening agent mixture is deposited in an anisotropic configuration, before crosslinking, followed by the exposure of the deposited alginate structure with cross-linkers such as calcium carbonate or D-Gluconic acid δ-lactone.

The microscale structure of the crosslinked printed object can be generated by controlled directional solidification freezing at block 20. Directional solidification freezing of the 3D printed biological matter freezes in such a way that ice crystals with controlled dimensions and orientations are produced. The purpose of the directional solidification process at block 20 is to generate controlled microstructure and texture in the 3D printed foods manufactured by directional 3D printing through controlled freezing. The directional freezing of the thin food slice, for example, can be either in a direction along the long part of the thin slice using directional solidification and a temperature gradient stage or in the direction of the thin slice thickness using a constant temperature stage.

The final product is removed from the substrate at block 22. The final product can be processed further by applying surface coatings such as flavorings or colorings, cooking and the object may also be reduced in size.

In the alternative embodiment 30 shown in FIG. 2, the biological or organic materials, thickening agent and crosslinking agent are selected at block 32 and the organic materials and thickening agent are mixed at block 34.

At block 36, the crosslinking agent is then added to the mixture of block 34 and the combination is quickly deposited or printed on the substrate before the crosslinking agent is fully activated at block 38. For example, in one embodiment, a sodium alginate thickening agent material is combined with cross-linkers such as calcium carbonate and D-Gluconic acid δ-lactone at block 36 prior to the 3D printing, followed by rapidly printing a macroscopic object at block 38 to generate an object with a controlled macrostructure.

The rapidly deposited structure is then frozen with directional solidification freezing at block 40 to create characteristic ice crystals with controlled dimensions and orientations to provide the desired microstructure. By controlling the direction, speed of motion and the temperature gradient to which the deposited structure is exposed, it is possible to control the direction and size of the ice crystals that form and provide control over the microscale anisotropic structure.

The ice is then removed and the final product is separated from the substrate at block 42. Additionally, the final product can also have some post-production applications of treatments such as flavorings or colors etc. at block 42.

In other embodiments, the sodium alginate thickening agent material is deposited before crosslinking in a macroscopic structure followed by the combination of the deposited alginate structure with cross-linkers such as calcium carbonate and D-Gluconic acid δ-lactone followed by directional solidification freezing.

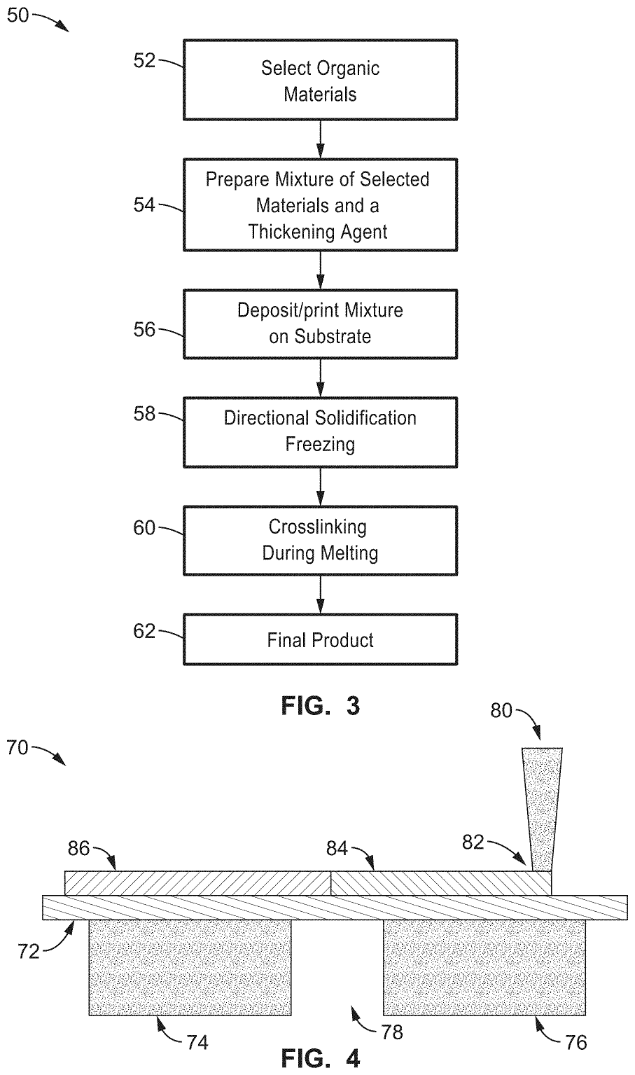

Another alternative embodiment of the method 50 is shown in FIG. 3. In this embodiment, the selected organic or biological materials at block 52 are mixed with the selected thickening agents at block 54 and the mixture is then deposited on a substrate at block 56. The produced mixture may be deposited on the substrate by directional 3D printing or by sequential deposition at block 56.

The deposited structure on the substrate from block 56 is then frozen with directional solidification at block 58. Ice crystals in the deposited material are in a desired orientation and with a desired size and these features are generated by controlling the temperature and heat flux during freezing.

A cross-linker is applied at block 60 to the frozen structure and the ice formed in frozen structure is allowed to melt. In another embodiment, the cross-linker is sprayed on the frozen structure during melting at block 60 and the crosslinking takes place during the ice melting.

For example, one embodiment with collagen used as the thickening agent, the desired macroscopic and microscopic anisotropic structure is achieved by freezing and generating ice crystals in the deposited material in a desired orientation and with a desired size, by controlling the temperature and heat flux during freezing and then immersing the frozen 3-D structure in a solution with the cross-linker at a controlled temperature so that the entire anisotropic structure becomes crosslinked as it thaws and the temperature becomes elevated. Crosslinking the printed product through the addition of the cross-linker as a spray on the surface of the printed product can be used with heat crosslinking for collagen or with UV crosslinking with collagen or with cross-linkers such as calcium carbonate and D-Gluconic acid δ-lactone for alginate. The final product is then removed and optionally post processing treatments are applied at block 62.

An object with desired macroscopic and microscopic anisotropic structure can also be achieved by freezing and generating ice crystals in the deposited alginate material in a desired orientation and with a desired size. The temperature and heat flux during freezing are controlled and then the frozen 3-D structure is immersed in a solution with cross-linkers such as calcium carbonate and D-Gluconic acid δ-lactone so that the entire anisotropic structure becomes crosslinked as it thaws and the cross-linker diffuses into the structure.

In another embodiment, the method comprises the steps of preparing a first solution of an aqueous solution or organic matter, forming the first solution into a plurality of two-dimensional individual anisotropic elements in parallel, each individual anisotropic element formed on a first surface, transferring the plurality of individual anisotropic elements to a second surface, assembling the plurality of individual anisotropic elements on the second surface in a three-dimensional array, and freezing the plurality of individual anisotropic elements in the three-dimensional array to complete the product.

The manufacturing of the products is preferably performed with a printing apparatus and technique that facilitates the deposition of volume elements with controlled anisotropic microscale structure. In contrast, conventional additive manufacturing of one 3D object of aqueous solutions and organic materials generally results in individual isotropic volume elements.

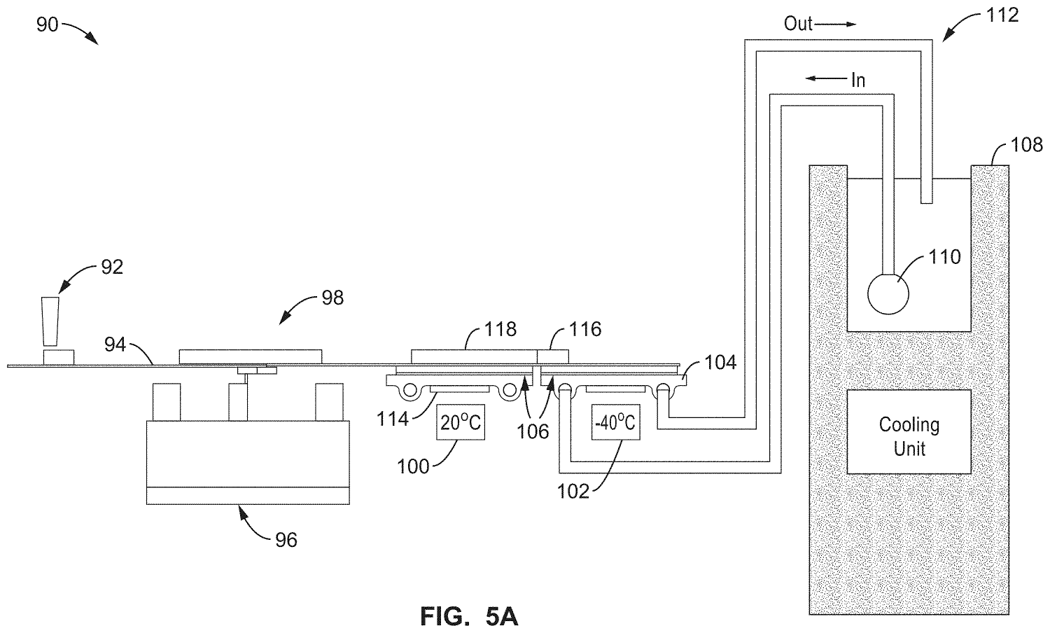

A simple printing apparatus 70 for 3D printing with directional solidification is shown schematically in FIG. 4. The parts of the 3D object are manufactured in a way that provides microscale anisotropic material properties and composition. The manufacturing process generally involves three elements: deposition of the 3D printing elements; cross-linking the material in the elements and between elements through chemical, thermal or electromagnetic means, and freezing the structure to generate desired ice crystals for a controlled microscale anisotropic texture. However, in some embodiments mechanical rigidity can be provided to aqueous and/or organic materials by freezing and drying the biological materials without crosslinking.

In the apparatus 70 embodiment shown in FIG. 4, a sliding platform 72 is provided that serves as the substrate for deposition or printing of organic material and is configured to move across the temperature controlled heating and cooling elements at a controlled rate. The sliding platform 72 is preferably thermally conductive. In an alternative embodiment, the sliding platform is placed on a conveyor or similar linear translation device and the printing surface is separate from the movement apparatus.

The thermally conductive sliding platform 72 is placed on a temperature controlled freezing element 74 that is below the freezing temperature of the liquids of the printed materials and a controlled heating element 76 with the two elements separated by an insulating gap 78 providing a thermal gradient. The sliding platform 72 moves across the warm and cold elements 74, 76 and gap 78 at a selected rate that will produce directional solidification of the printed materials.

A print head 80 dispenses a layer of material 82 on to the top printing surface of the sliding platform 72. The printed material can be applied according to a pattern by a controller and programming (not shown) that controls the position of the print head 80 and the timing and volume of material 82 that is dispensed. Although a single printer dispensing head 80 is shown delivering a single type of material, it will be understood that the printing head 80 can dispense more than one type of material in the formation of the 3D object. Multiple print heads 80 may also be used for the controlled deposit of material. In various embodiments, the deposited material may be a paste, dispersion, suspension, slurry or solution of organic matter that is printed in layered patterns as determined by the printer programming.

A sprayer that delivers the cross-linker 84 can be positioned between the 3D printer head 80 and the location of the directional freezing device 74 or after the directional freezing device. In one embodiment, the spray 86 of cross-linker is applied to the frozen material and crosslinks the structure as the material thaws as illustrated in the embodiment of FIG. 3.

The material 82 to be frozen that is placed on top of the surface of the sliding platform 72 by the print head 80 will attain the temperature of the platform surface or previous layer, which can vary from a temperature above freezing to below freezing. When the substrate platform 72 is pushed with a given velocity across the temperature gradient produced by the two temperature controlled warm and cold module elements 74, 76, the material on the surface freezes with a controlled velocity and the ice crystals form in the direction of the movement of the surface (i.e. directional solidification). This facilitates continuous printing and directional freezing of thin food samples with controlled anisotropic microscale structure, for example.

Turning now to FIG. 5A and FIG. 5B, the systems and methods 90 may further perform the manufacture of 3D objects of aqueous solutions and organic materials in a parallel form, such that all the steps of the additive manufacturing are not performed sequentially at one station (as in conventional additive manufacturing) but rather in at least two stations where the steps can be performed in parallel. These systems and methods 90 can facilitate large scale additive manufacturing of 3D objects made of aqueous solutions and/or organic materials by operating in parallel, thereby reducing the time of manufacturing of the 3D objects.

In this illustration, the deposition, cross-linking and directional solidification stages are arranged in parallel as seen in the top view of FIG. 5B. In the printing stage, the printer head 92 of each lane deposits a film or pattern of material on the slide platform 94 or separate printing platform on top of the slide platform. The slide platform 94 can be moved from the printing stage through the cross-linking and directional solidification stages with a linear translation system 96 such as a conveyor belt. The linear translation system 96 controls the rate of linear translation across the heating and cooling elements of the apparatus.

After printing, the cross-linker 98 can be applied by a sprayer or other delivery system to stabilize the printed structure in this embodiment. The crosslinked printed material on the printing platform 94 is then moved through the directional solidification stage. This stage has a warm module and a cold module separated by a gap positioned under the printing platform. In the side view of FIG. 5A it can be seen that the warm side has a temperature sensor, element and controller 100 and a cold temperature sensor, element and controller 102. The warm side temperature controller 100 modulates the temperature of the slide platform 94 and crosslinked printed material with resistive film heater 106 heating elements in this embodiment.

The cooling module of the directional solidification stage has a cooling element 104 positioned under the platform that is connected to a cooling unit 108. In this embodiment, the cooling unit 108 chills methanol that is pumped through the input and output ducts 112 with a submersible pump 110. The cooling element 104 and gap will freeze the printed material with a controlled freeze as the platform moves over the heating and cooling elements. As seen in FIG. 5B, the cross-linked printed material 118 will be progressively frozen 116 at a controlled rate as the object traverses the heating and cooling modules. The optional post-processing stage 120 may take place after the directional solidification stage. Post processing 120 may include sublimation or melting of the frozen liquids as well as the application of flavors or colors or other treatments to the final product.

In the embodiment illustrated in FIG. 5A, the slide platform 94 may be made from a thin sheet of metal such as stainless steel. An optional permanent or electromagnet 114 may be positioned under the heating 100 and cooling 102 modules to increase platform contact and reduce the thermal contact resistance between the platform 94 and the modules to improve thermal efficiencies and to equalize the cooling or heating across the platform 94.

In one embodiment, the rate of freezing, heating and cooling element temperatures, platform movement and printing are all controlled by a controller with programming software. Controller programming allows fine control over the volume, size and orientation of the deposited materials, the timing of crosslinking and control over the temperature, heat flux during freezing and freezing rate that all play an important role in the design of the macro and microscale structure of the final object.

The technology described herein may be better understood with reference to the accompanying examples, which are intended for purposes of illustration only and should not be construed as in any sense limiting the scope of the technology described herein as defined in the claims appended hereto.

### Example 1

In order to demonstrate the functionality of the systems and methods, an apparatus like that shown generally in FIG. 4 was fabricated and tested to demonstrate anisotropic printing on a moving surface. A commercial 3-D printer (Flashforge USA, Los Angeles, Calif.) was modified with new hardware for easier control of print settings and the extruder was redesigned to allow for printing of highly viscous biomaterials. A material that was viscous enough to maintain the structure temporarily, before cross linking or freezing, was evaluated.

The material used in this example was a finely pureed beef muscle mixed at a ratio of volumes of 5:1 with a solution of 4% w/v sodium alginate hydrogel prepared by mixing the nominal mass of sodium alginate (Molecular Weight: 222 g/mol) (Spectrum Chemical Mfg. Corp., Gardena, Calif.) in the nominal volume of distilled water (Fisher Science Education, Nazareth, Pa.). The mixture was stirred on a magnetic stir plate until homogenous.

In this example, the extruder was a syringe. However, other devices for delivering the printed material were evaluated including a continuous pump and an Archimedes screw. To allow for the printing of liquids and gels from a syringe, the motor was mounted above the syringe and aligned with the syringe plunger so as to apply an axial force on the plunger with minimal movement. The motor was also placed on the moving carriage with its back face in direct contact with the syringe plunger, so that its weight facilitates the pushing of the syringe. This design resulted in the lead screw extending upwards out of the extruder with the motor being driven in reverse to extrude from the syringe and forward driven to retract.

The print platform was also reconfigured to facilitate the manufacturing of thin large food objects. It consisted of a surface that could be moved in relation to the 3-D printer. In this design, for example, the movement along the axis of the printing surface (x) could be accomplished by the movement of the printing surface, while the movement along the axis normal to x, y was accomplished by moving the printer head. In contrast in conventional 3-D printing, the head moved in the (x-y) direction and the printing surface moved only in the (z), direction. Here the printing surface could move in the (x) and (z) direction.

In all the examples, the shape to be printed was designed in a 3D CAD software (Onshape, Cambridge, Mass.) and then exported as a STL file to be sliced into elements in a 3D print slicing application, Ultimaker Cura (Ultimaker B.V., The Netherlands) which converted the 3D models into instructions and (x), (y), (z) movements, known as G-code, which a printer can then interpret. The programmed velocity of the head was designed to facilitate a continuous deposition of the material along the direction of the anisotropic element central axis, rather than a voxel-by-voxel deposition.

This apparatus was used to deposit the 3D elements to form several large thin objects. Thereafter, the printed object was sprayed with a 1% w/v CaCl₂) solution to crosslink the object as the cross-linker diffused into the material. The crosslinking solution was prepared by mixing the appropriate mass of CaCl₂) dihydrate (Fisher Scientific, Fair Lawn, N.J.) in distilled water. This step could be done immediately after the printing of the object or after freezing of the object and during the thawing of the object. Heat can be used in a similar way to the chemical cross-linker in the case of a material made with collagen. The heat was applied to the outer surface of the object and the heat driven crosslinking process diffused into the object.

The resulting food object was boiled to evaluate the integrity of the manufactured food object. It was observed that the integrity of the object was maintained and it could qualify as a food product consisting of a thin slice of meat. This could be of value for food in the Far East cuisine, for example, where thin slices of meat are regularly consumed.

In another printing with this apparatus, the finely pureed beef muscle mixed at a ratio of volumes of 5:1 with a solution of 4% w/v sodium alginate hydrogel was prepared as above. The composition of the alginate and beef muscle was designed to be viscous enough to withstand the printing. Several anisotropic layers could be printed one on top of the other, provided that the viscosity was high enough to retain the structure. A finished thin multilayer object was fabricated. After the printing, the object was immersed in a solution of in a 1% w/v CaCl₂) solution to crosslink as the cross-linker diffused into the object. This step was done immediately after the printing of the 3-D object in one test and after freezing of the object during the thawing of the object in another.

### Example 2

To further demonstrate the functionality of additive manufacturing systems and methods, a directional solidification device with parallel processing was assembled and tested. The apparatus, shown schematically in FIG. 5A and FIG. 5B, was used to perform directional solidification (freezing) on a thin slice of a food product, manufactured with additive manufacturing to obtain after melting a desired texture through the controlled anisotropic microstructure formed by freezing.

The apparatus had three printing stages and directional solidification stages arranged in parallel as illustrated in FIG. 5B. The cooling module consisted of two Microcool CP3001 cold plates (Wieland Microcool, Decatur, Ala.). These cold plates were hollow to allow fluid circulation between finned microchannels for rapid heat exchange at their surfaces. Mounted to each cold plate were three 120 mm×60 mm customized aluminum plates, with grooves to keep the stainless-steel slide moving in a linear path. Translating along these grooves was a stainless-steel plate on which the liquid sample is placed, contained edgewise with a walled perimeter. Stainless steel was selected for its relatively high thermal conductivity and magnetic properties. A strong, permanent magnet was positioned under each of the temperature-regulated modules to attract the steel to the module and reduce thermal contact resistance. The walled perimeter was shaped to control the origin and direction of ice nucleation.

Between the cold plates and the aluminum plates were Omega KHA-106 thin film polyimide heaters (Omega Engineering, Norwalk, Conn.), working in conjunction with Omega Platinum Series CN32PT-220 temperature controllers (Omega Engineering, Norwalk, Conn.) and Omega Type J thermocouples (Omega Engineering, Norwalk, Conn.) to regulate surface temperatures. The thermocouples were placed on the leading edge of the aluminum blocks to ensure controlled cooling within the gap. Methanol at dry ice temperature (approximately −78° C.) was circulated through the low temperature cold plate using a submersible pump. The cold side controller regulated the methanol pump and thin film heater based on input from the thermocouple to keep it at the cold temperature while the hot side controller regulated only a thin film heater. This system provided temperature regulation within ±0.3° C. of the setpoint.

### Example 3

Ice formation and microstructure using directional solidification by controlled freezing of biomaterials were evaluated to demonstrate the capability of the methods to control product morphology. Ice morphology, hydrogel microstructure, mechanical properties, and crosslinking were observed and analyzed from directional freezing.

Before evaluating the effect of cross-linking on microstructure, differences in ice morphology in crosslinked and non-crosslinked alginate were investigated, since ice crystals can dictate microstructure in frozen hydrogel. Samples of alginate that were part crosslinked and part non-crosslinked were frozen under continuous monitoring and the ice crystal morphology in crosslinked alginate was compared to the ice crystal morphology in non-crosslinked alginate frozen under identical conditions. The crystal morphology was evaluated by optical microscopy.

First, a 2% sodium alginate solution was prepared by adding the appropriate amount of sodium alginate powder (molecular weight: 222 g/mol) (Spectrum Chemical Mfg. Corp. Gardena, Calif.) to deionized (DI) water. The solution was then mixed on a magnetic stir plate until homogenous and then allowed to settle in the refrigerator (set to 4° C.) for at least 24 hours before use. For crosslinking, a 2% w/v solution of calcium chloride (CaCl₂) was made by adding 4 g of CaCl₂ dihydrate powder (Fisher Scientific, Fairlawn, N.J.) to 200 ml of water and the resulting CaCl₂ solution contained in an atomizer spray bottle.

Second, the alginate solution was poured into a 0.75 mm sample holder until full, for a sample thickness of 0.75 mm. To make part of the sample crosslinked, a portion (approximately the first half of the length) of the solution cast in the sample holder was covered with a microscope slide and the uncovered length of the sample was sprayed with the 2% CaCl₂ solution to initiate crosslinking. The resulting sample was half crosslinked and half non-crosslinked. The sample was then frozen directionally on the directional device, beginning with the non-crosslinked portion, until the entire sample was frozen. In these illustrations, the high temperature surface of the directional device was set to 40° C. and the low temperature surface to −40° C. A 4 mm gap in between them was maintained using 4 microscope slides, each 1 mm thick placed in between the plates, for a resulting temperature gradient of 20° C./mm. A cooling rate of 120° C./min was achieved by moving the sample at a velocity of 6 mm/min using a modified syringe pump (Harvard Apparatus, Holliston, Mass.) to translate the stainless-steel slide. The morphological appearance of the ice crystals was monitored with the microscope and recorded.

The ice crystal morphology at the freezing interface was captured using an Olympus SZ61 light microscope at 8× magnification (Olympus Corporation, Tokyo, Japan) placed above the directional device, focused on the gap between the high and low temperature modules, as shown in FIG. 4. Freezing of each sample was recorded using an Olympus C-7070 digital camera (Olympus Corporation, Tokyo, Japan) as the sample traversed the temperature gradient across the gap.

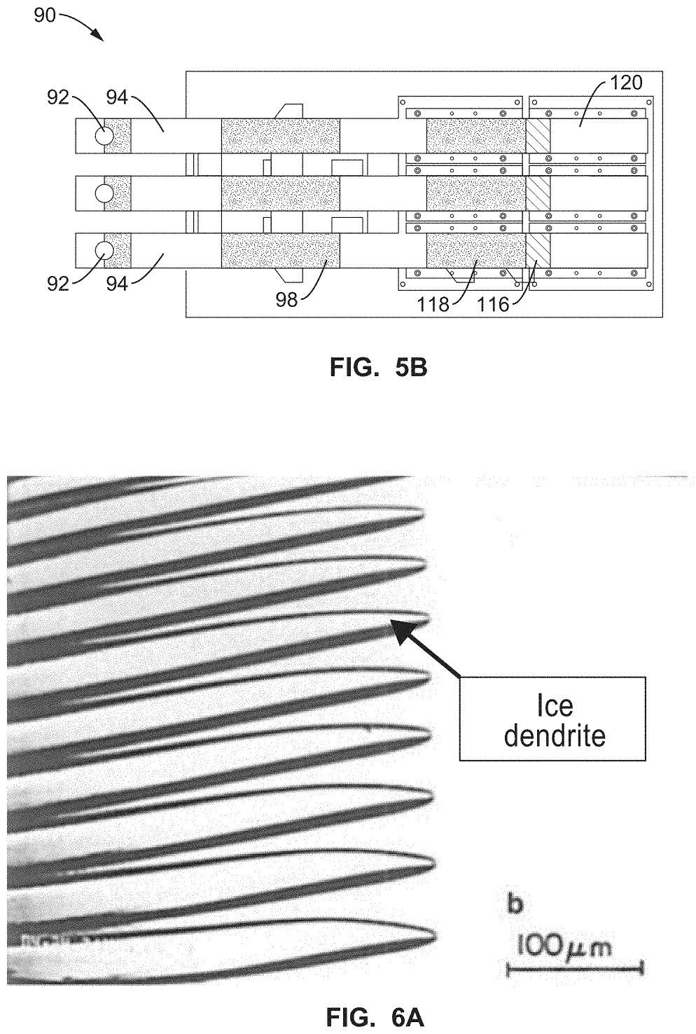

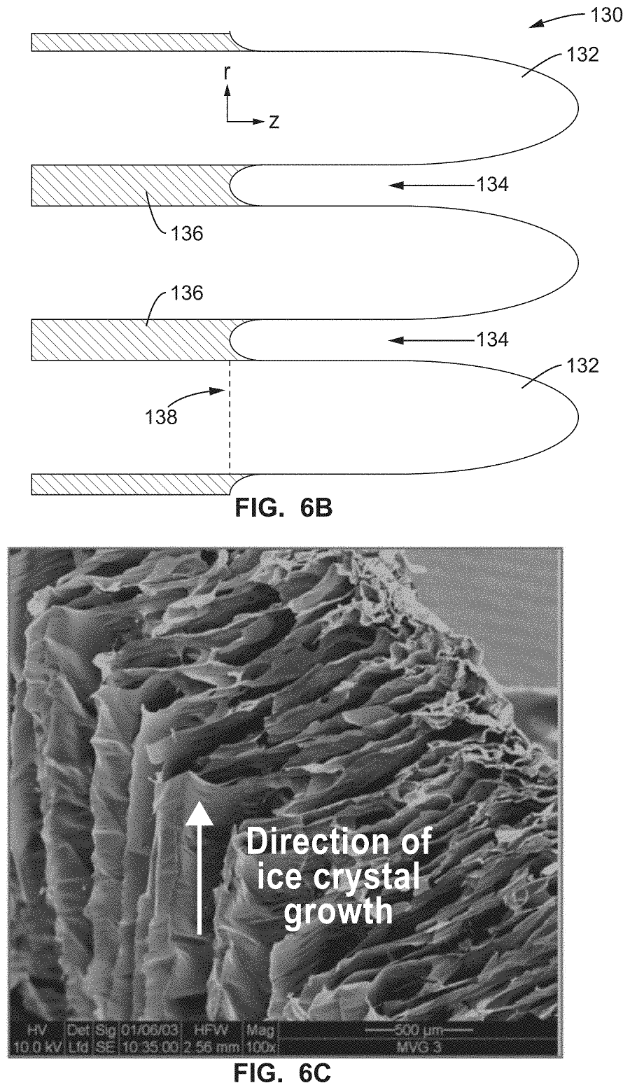

The typical outcome of directional solidification of an aqueous saline solution, seen under an optical microscope, is shown in FIG. 6A. The directional ice structure after directional freezing had repeating ice dendrites with a thickness of about 50 μm. The morphology of the dendritic ice crystal structure 130 is shown schematically in FIG. 6B. The ice dendrites 132 are directional and substantially in parallel. Between the pure ice dendrites 132 is an inter-dendritic liquid 134 and the eutectic solid material 136. The basal plane 138 of a dendrite is shown as a dashed line in FIG. 6B.

FIG. 6C shows the outcome of a directional solidification freezing of an agar gel solution, as seen after freeze drying and scanning electron microscopy. Note the directionality of the voids formed by ice sublimation.

The differences in the ice crystal morphology in the non-crosslinked region compared to the crosslinked region during freezing, as observed by optical microscopy, were strongly dependent on the temperature gradients during freezing and freezing rates. As the cooling rates and the freezing velocity increase, the size of the ice crystal decreases, respectively.

However, for these parameters, freezing in a non-crosslinked alginate resulted in continuous parallel ice crystals. Freezing in a crosslinked alginate, while still directional, had lost the straight structure of the ice crystals. It was obvious that the thermal parameters play an important role in the design of the microscale structure.

### Example 4

The effect of the crosslinking to various densities before freezing was investigated with electron microscopy. Having found an effect of crosslinking before freezing on the ability to form directional microstructure in alginate hydrogels, an additional evaluation of the influence of crosslinking density was performed. The threshold for crosslinking density before directional microstructure as a result of ice dendrite formation is impeded in alginate was identified.

A 2.4% w/v sodium alginate solution was first prepared and multiple aqueous calcium carbonate (CaCO₃) (Acros Organics, New Jersey) or D-(+)-Gluconic acid S-lactone (GDL) (Sigma-Aldrich Co, St. Louis, Mo.) solutions were mixed individually. Calcium carbonate was used, as opposed to CaCl₂), for its low crosslinking rate due to high pH in the absence of GDL, which permitted greater homogeneity in the crosslinked mixture. GDL was used to buffer the alkalinity of calcium carbonate, allowing quick crosslinking after calcium ion incorporation. The alkalinity of the CaCO₃ solution prevents the dissolution of the CaCO₃ and limits the Ca²⁺ ions available for crosslinking. The concentrations of the CaCO₃ solutions were determined under the assumption that one Ca²⁺ ion would bind two sodium alginate carboxyl groups. This saturated molar ratio was designated as 1× cross-linking, for notation.

Three CaCO₃ solutions were prepared such that when mixed with a specific volume of alginate would dilute it to 2% w/v. Furthermore, their calcium ion molarities would crosslink the alginate to 0.125×, 0.0625×, and 0.03125× (where × signifies the multiplier for the saturated molar ratio designated 1×). These values were chosen to maintain fluidity during processing as well as structure after freeze drying, while providing substantial crosslinking differences for comparison. Mixing CaCO₃ with alginate involved constant resuspension, gradual micro pipetting and stirring for 20 minutes.

After forming three aqueous sodium alginate and CaCO₃ suspensions, aqueous GDL was incorporated. GDL was added in equal volume to CaCO₃, providing a 2:1 GDL to CaCO₃ molar ratio to each suspension. Mixing GDL involved gradual micro-pipetting and stirring for 20 minutes to prevent clusters. Once thoroughly stirred, each solution was stored in a 4° C. refrigerator for at least 24 hours.

Each solution was deposited into a 5 mm thick sample holder resting on the stainless-steel substrate. This larger sample thickness allowed easier removal of the sample after freezing and better structural integrity after freeze drying. A cooling rate of 60° C./min was achieved in the directional device by setting the high temperature surface to 40° C., the low temperature surface to −40° C., maintaining a 4 mm gap between the two surfaces, and moving the samples 3 mm/min. Each sample was frozen across the directional device as previously described. Once frozen, each sample was removed from the mold and freeze dried for 24 hours using a Martin Christ Alpha 1-2 1d freeze dryer (Martin Christ, Germany). This process was repeated twice for each of the three crosslinking densities, resulting in six samples.

Micrographs were obtained of each crosslinking density using a Hitachi TM-1000 scanning electron microscope (SEM) at 15 kV. Images of cross sections perpendicular to the direction of ice growth (transverse), cross sections parallel to the direction of ice growth (lateral), and top surfaces were obtained at various magnifications. The results depict a progression from an aligned structure to a more random structure as the crosslinking density was increased.

At a 0.03125× cross-linking density the pores appear aligned and directed parallel to the direction of the temperature gradient. This is similar in structure to the samples crosslinked after freezing suggesting a lightly crosslinked structure has enough free water molecules for the continuous attachment to ice dendrites. Similarly, at a 0.0625× crosslinking density there was still alignment in the lateral and top cross sections, although there is greater non-uniformity in the pore sizes in the transverse section.

At a crosslinking density of 0.125×, there was a clear loss of directional alignment similar to samples crosslinked before freezing. There was no significant difference in the microstructure in the transverse and lateral cross sections of the sample with the highest crosslinking density suggesting a lack of pore alignment, unlike in lower crosslinking density samples. At the higher crosslinking density, the directional dendritic ice crystals could not develop as in the lower cross-linking densities.

### Example 5

The effect of directional microstructure on the mechanical properties of the resulting alginate hydrogel made using directional solidification was evaluated. The aligned anisotropic microstructure shown earlier suggests similar anisotropy in the mechanical properties of the alginate gel. Tensile tests were conducted to evaluate the mechanical properties of different compositions of alginate, comparing those crosslinked before freezing (which have isotropic microstructure) to those crosslinked after freezing (which have aligned anisotropic microstructure).

From each directional frozen sample, up to four mechanical testing strips about 40 mm long by 10 mm samples were obtained. Three strips were cut with 40 mm lengthwise dimension along the grains of the ice crystal growth and one strip across the ice crystal growth dimension. Each test strip was then loaded onto a Shimadzu Universal Tensile test machine (Shimadzu Corp, Tokyo, Japan) with a 100N load cell. All tests were done at a fixed strain rate of 50 mm/min.

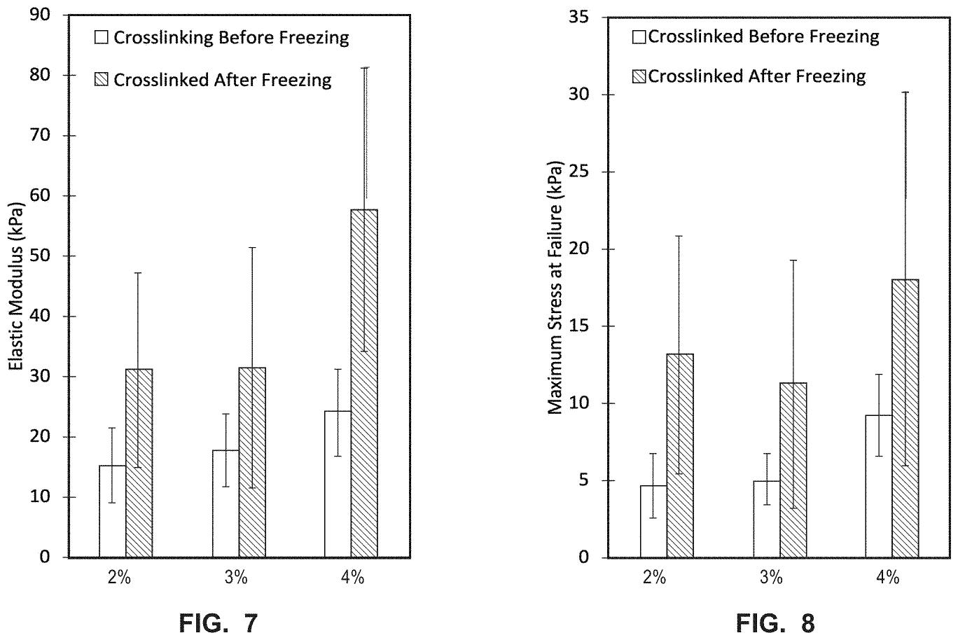

The elastic moduli of the tested samples crosslinked before freezing is compared with those crosslinked after freezing for various compositions is shown in FIG. 7. The mean elastic moduli of the samples crosslinked before freezing were 15.3 kPa, 17.9 kPa, and 24.1 kPa for 2%, 3% and 4% w/v alginate solutions respectively. The respective means of the samples crosslinked after freezing were 31.1 kPa, 31.6 kPa, and 57.7 kPa. Although, the mean elastic modulus increases going from the non-aligned microstructure in the samples crosslinked before freezing to the aligned microstructure in the samples crosslinked after freezing, statistical analysis showed no significant difference in the means.

Maximum stress at failure of the samples crosslinked before freezing as compared with those crosslinked after freezing at different compositions of alginate is shown in FIG. 8. The mean stresses at failure of the samples crosslinked before freezing were 4.7 kPa, 5.0 kPa, and 9.2 kPa for 2%, 3% and 4% alginate solutions respectively. The mean stresses at failure of the samples crosslinked after freezing were 13.2 kPa, 11.3 kPa, 18.1 kPa respectively. There was also no significant difference in the mean failure stresses between those crosslinked before freezing and those cross-linked after freezing.

It can be seen that there are various parameters involved in the manufacturing and the composition of the directionally solidified agarose which can affect the microscale structure of the product as well as the mechanical properties of the product. Therefore, food properties can be adjusted and optimized through the judicious selection of fabrication parameters.

From the description herein, it will be appreciated that the present disclosure encompasses multiple implementations which include, but are not limited to, the following:

A method of additive manufacturing objects of biological matter, the method comprising: (a) preparing a deposition mixture of an aqueous composition of biological matter and a thickening agent; (b) iteratively depositing the deposition mixture on a substrate to form an object; (c) applying a cross-linker to the object to produce a crosslinked object; and (d) freezing the crosslinked object with directional solidification to generate an anisotropic microstructure in the crosslinked object.

The method of any preceding or following implementation, wherein the biological material is at least one material selected from the group of plant tissue particles, animal tissue particles, a protein, a fat, and a carbohydrate.

The method of any preceding or following implementation, wherein the thickening agent is an agent selected from the group of agents consisting of agar, collagen, and an alginate.

The method of any preceding or following implementation, wherein the cross-linker is selected from the group of cross-linkers consisting of chemical, thermal and electromagnetic cross-linkers.

The method of any preceding or following implementation, wherein the cross-linker is selected from the group of cross-linkers consisting of calcium carbonate or D-Gluconic acid δ-lactone.

The method of any preceding or following implementation, further comprising drying the frozen object by thawing the frozen object and evaporating water.

The method of any preceding or following implementation, further comprising drying the frozen object by sublimation of ice crystals.

A method of additive manufacturing of biological matter, the method comprising: (a) preparing a deposition mixture of an aqueous composition of biological matter and a thickening agent; (b) adding a cross-linker to the deposition mixture; (c) rapidly depositing the deposition mixture with cross-linker on to a substrate to form a crosslinked object; and (d) freezing the crosslinked object with directional solidification to generate an anisotropic microstructure in the crosslinked object.

The method of any preceding or following implementation, wherein the biological material is at least one material selected from the group of plant tissue particles, animal tissue particles, a protein, a fat, and a carbohydrate.

The method of any preceding or following implementation, wherein the thickening agent is an agent selected from the group of agents consisting of agar, collagen, and an alginate.

The method of any preceding or following implementation, wherein the cross-linker is selected from the group of cross-linkers consisting of calcium carbonate or D-Gluconic acid δ-lactone.

A method of additive manufacturing of biological matter, the method comprising: (a) preparing a deposition mixture of an aqueous composition of biological matter and a thickening agent; (b) iteratively depositing the deposition mixture on a substrate to form an object; (c) freezing the deposited object with directional solidification to generate an anisotropic microstructure in the frozen deposited object; (d) thawing the frozen deposited object; (e) applying a cross-linker to the frozen object while thawing to produce a crosslinked object; and (f) drying the crosslinked object by evaporation.

The method of any preceding or following implementation, wherein the biological material is at least one material selected from the group of plant tissue particles, animal tissue particles, a protein, a fat, and a carbohydrate.

The method of any preceding or following implementation, wherein the thickening agent is an agent selected from the group of agents consisting of agar, collagen, and an alginate.

The method of any preceding or following implementation, wherein the cross-linker is selected from the group of cross-linkers consisting of calcium carbonate or D-Gluconic acid δ-lactone.

An apparatus for additive manufacturing of biological matter objects, the apparatus comprising: (a) a printing head assembly with a dispenser configured for dispensing an aqueous deposition mixture on to a printing surface of a substrate; (b) a linear translation platform supporting the substrate, the linear translation platform configured for positional translation of the linear translation platform and substrate at a controlled rate; (c) a sprayer system configured to spray a cross-linker solution on a printed deposition mixture on the substrate; (d) a directional solidification stage of a warm module and a cold module separated by a gap, wherein movement of the substrate across the warm and cold modules and gap creates a temperature gradient in the printed deposition mixture and substrate; and (e) a control mechanism operably coupled to the printing head assembly, linear translation platform, sprayer system and directional solidification stage, the control mechanism configured for: (i) positioning the printing head assembly and printing the deposition mixture onto the printing surface of the substrate to form a printed deposition mixture; (ii) controlling the position of the linear translation platform and substrate; (iii) operating the sprayer system to spray cross-linker solution on the printed deposition mixture on the substrate; (iv) moving the linear translation platform and substrate across the warm module, gap and cold module at a controlled rate; and (v) controlling temperatures of the cold module and the warm module; (vi) wherein directional solidification of the printed deposition mixture produces a product with anisotropic microstructure.

The apparatus of any preceding or following implementation, wherein the sprayer system sprays cross-linker after the directional solidification stage.

The apparatus of any preceding or following implementation, wherein the warm module comprises: a thermally conductive plate; a thin film heater attached to the thermally conductive plate; and at least one temperature sensor.

The apparatus of any preceding or following implementation, wherein the cold module comprises: a thermally conductive plate with a plurality of ducts; a cooling unit chilling a liquid to a temperature below the freezing temperature of water and pumping chilled liquid through the ducts of the thermally conductive plate; an optional thin film heater attached to the thermally conductive plate; and at least one temperature sensor.

The apparatus of any preceding or following implementation, further comprising: a plurality of printing head assemblies, linear translation platforms, sprayers and directional solidification stages aligned in parallel; one or more processors operably coupled to the aligned printing head assemblies, linear translation platforms, sprayers and directional solidification stages; and a non-transitory memory storing executable instructions that, if executed by the one or more processors, configure the apparatus to: positioning each printing head assembly and printing the deposition mixture onto the printing surface of the substrate to form a printed deposition mixture; controlling the position of each linear translation platform and substrate; operating each sprayer system to spray cross-linker solution on the printed deposition mixture on the substrate; moving each linear translation platform and substrate across the warm module, gap and cold module at a controlled rate; and controlling temperatures of each cold module and each warm module.

As used herein, term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these group elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system or method.

As used herein, the term “set” refers to a collection of one or more objects. Thus, for example, a set of objects can include a single object or multiple objects.

Relational terms such as first and second, top and bottom, and the like may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential features or elements of the technology describes herein or any or all the claims.

In addition, in the foregoing disclosure various features may grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after that application is filed. Accordingly the reader should consult the application as filed for the original content of the disclosure. Any deletion of content of the disclosure should not be construed as a disclaimer, forfeiture or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A method of additive manufacturing objects of biological matter, the method comprising:
(a) preparing a deposition mixture of an aqueous composition of biological matter and a thickening agent;
(b) iteratively depositing the deposition mixture on a substrate to form an object;
(c) applying a cross-linker to the object to produce a crosslinked object; and
(d) freezing the crosslinked object with directional solidification to generate an anisotropic microstructure in the crosslinked object.

2. The method of claim 1, wherein said biological material is at least one material selected from the group of plant tissue particles, animal tissue particles, a protein, a fat, and a carbohydrate.

3. The method of claim 1, wherein said thickening agent is an agent selected from the group of agents consisting of agar, collagen, and an alginate.

4. The method of claim 1, wherein said cross-linker is selected from the group of cross-linkers consisting of chemical, thermal and electromagnetic cross-linkers.

5. The method of claim 1, wherein said cross-linker is selected from the group of cross-linkers consisting of calcium carbonate or D-Gluconic acid δ-lactone.

6. The method of claim 1, further comprising drying the frozen object by thawing the frozen object and evaporating water.

7. The method of claim 1, further comprising drying the frozen object by sublimation of ice crystals.

8. A method of additive manufacturing of biological matter, the method comprising:
(a) preparing a deposition mixture of an aqueous composition of biological matter and a thickening agent;
(b) adding a cross-linker to the deposition mixture;
(c) rapidly depositing the deposition mixture with cross-linker on to a substrate to form a crosslinked object; and
(d) freezing the crosslinked object with directional solidification to generate an anisotropic microstructure in the crosslinked object.

9. The method of claim 8, wherein said biological material is at least one material selected from the group of plant tissue particles, animal tissue particles, a protein, a fat, and a carbohydrate.

10. The method of claim 8, wherein said thickening agent is an agent selected from the group of agents consisting of agar, collagen, and an alginate.

11. The method of claim 8, wherein said cross-linker is selected from the group of cross-linkers consisting of calcium carbonate or D-Gluconic acid δ-lactone.

12. A method of additive manufacturing of biological matter, the method comprising:
(a) preparing a deposition mixture of an aqueous composition of biological matter and a thickening agent;
(b) iteratively depositing the deposition mixture on a substrate to form an object;
(c) freezing the deposited object with directional solidification to generate an anisotropic microstructure in the frozen deposited object;
(d) thawing the frozen deposited object;
(e) applying a cross-linker to the frozen object while thawing to produce a crosslinked object; and
(f) drying the crosslinked object by evaporation.

13. The method of claim 12, wherein said biological material is at least one material selected from the group consisting of plant tissue particles, animal tissue particles, a protein, a fat, and a carbohydrate.

14. The method of claim 12, wherein said thickening agent is an agent selected from the group of agents consisting of agar, collagen, and an alginate.

15. The method of claim 12, wherein said cross-linker is selected from the group of cross-linkers consisting of calcium carbonate or D-Gluconic acid δ-lactone.

16. An apparatus for additive manufacturing of biological matter objects, the apparatus comprising:
(a) a printing head assembly with a dispenser configured for dispensing an aqueous deposition mixture on to a printing surface of a substrate;
(b) a linear translation platform supporting the substrate, the linear translation platform configured for positional translation of the linear translation platform and substrate at a controlled rate;
(c) a sprayer system configured to spray a cross-linker solution on a printed deposition mixture on the substrate;
(d) a directional solidification stage of a warm module and a cold module separated by a gap, wherein movement of the substrate across the warm and cold modules and gap creates a temperature gradient in the printed deposition mixture and substrate; and
(e) a control mechanism operably coupled to the printing head assembly, linear translation platform, sprayer system and directional solidification stage, the control mechanism configured for:
(i) positioning the printing head assembly and printing the deposition mixture onto the printing surface of the substrate to form a printed deposition mixture;
(ii) controlling the position of the linear translation platform and substrate;
(iii) operating the sprayer system to spray cross-linker solution on the printed deposition mixture on the substrate;
(iv) moving the linear translation platform and substrate across the warm module, gap and cold module at a controlled rate; and
(v) controlling temperatures of the cold module and the warm module;
(vi) wherein directional solidification of the printed deposition mixture produces a product with anisotropic microstructure.

17. The apparatus of claim 16, wherein said sprayer system sprays cross-linker after the directional solidification stage.

18. The apparatus of claim 16, wherein said warm module comprises:
a thermally conductive plate;
a thin film heater attached to the thermally conductive plate; and
at least one temperature sensor.

19. The apparatus of claim 16, wherein said cold module comprises:
a thermally conductive plate with a plurality of ducts;
a cooling unit chilling a liquid to a temperature below the freezing temperature of water and pumping chilled liquid through said ducts of said thermally conductive plate;
an optional thin film heater attached to the thermally conductive plate; and
at least one temperature sensor.

20. The apparatus of claim 16, further comprising:
a plurality of printing head assemblies, linear translation platforms, sprayers and directional solidification stages aligned in parallel;
one or more processors operably coupled to said aligned printing head assemblies, linear translation platforms, sprayers and directional solidification stages; and
a non-transitory memory storing executable instructions that, if executed by the one or more processors, configure the apparatus to:
positioning each printing head assembly and printing the deposition mixture onto the printing surface of the substrate to form a printed deposition mixture;
controlling the position of each linear translation platform and substrate;
operating each sprayer system to spray cross-linker solution on the printed deposition mixture on the substrate;
moving each linear translation platform and substrate across the warm module, gap and cold module at a controlled rate; and
controlling temperatures of each cold module and each warm module.
